(19) United States
(12) Patent Application Publication
(10) Pub. No.: US 2006/0271092 A1
(43) Pub. Date: Nov. 30, 2006

(54) Balloon-in-balloon cervical canal dilator

(76) Inventors: David Reed, New Canaan, CT (US); Jonathan Foltz, Wilton, CT (US)

Correspondence Address: Stephen E. Feldman, P.C., 12 East 41st Street, New York, NY, 10017, US

(21) Appl. No.: 11/140,323

(22) Filed: May 27, 2005

(51) Int. Cl. A61M 29/00

(52) U.S. Cl. 606/193

(57) ABSTRACT

A cervical canal dilator having an elongate tubular shaft that defines at least two internal lumens that are juxtaposed to a longitudinal axis. A first inflatable member is positioned on the outer surface of the shaft and is in communication with the first lumen. While a second inflatable member is also positioned on the outer surface of the shaft. The second inflatable, however, is longer than and completely covers the first inflatable member. Additionally, the second member is fabricated of a non-elastic material that limits the inflation of the second member to a predetermined maximum diameter of inflation. The device, also, has a control system which controls the fluids and/or gases that enter the lumens.

### THE FIELD OF THE INVENTION

This particular invention generally pertains to a cervical canal dilator having an elongate tubular shaft that defines at least two internal lumens which are juxtaposed to a longitudinal axis. More specifically, it is directed to a dilator having a first inflatable member and a second inflatable member wherein the second member is longer than and completely covers the first inflatable member.

### BACKGROUND OF THE INVENTION

Prior inelastic balloon designs for cervical dilation have been inadequate due to the lack of visualization of the cervical canal to assist the user in placing the catheter. This differs from the use of inelastic balloons in angioplasty; when balloons are used in angioplasty the catheter placement is visualized with fluoroscopy. For this reason, a single balloon for dilation, similar to that used in angioplasty, is ineffective, resulting in insertion of the catheter too far, causing damage to the uterus, or not inserting the catheter far enough, causing failure to dilate the full length of the cervix. This problem is exacerbated by the uneven resistance along the length of the cervical canal.

There is significantly more resistance to dilation at the internal os (the distal portion of the cervical canal at the entrance to the uterine cavity) than the portion of the cervix closer to the vagina. If dilation is attempted with a catheter using a single non-compliant (non-elastic) balloon, similar to a traditional angioplasty catheter, and the balloon is not properly positioned with the distal tip well beyond the inner os, the higher resistance at the inner os, the portion of the cervix closest to the uterus, will push the tapered balloon tip outward, resulting in incomplete dilation.

Several alternative designs to correct this deficiency do not adequately address the issues surrounding dilation of the inner os. These alternatives will potentially result in insufficient dilation of some portion of the cervix or over dilation of a portion of the cervix, risking damage to the cervix that could lead to cervical incompetence later.

One of the obvious possible alternative designs is to lengthen the balloon to allow placement of the distal balloon edge well past the inner os to reduce the potential for the balloon to be squeezed out. The failing of this potential design is that the distal tip of the catheter must be located within the body of the uterus. The length of the cervix and depth of the uterine cavity vary by several centimeters between patients and the actual length of the cervical canal is not known in most patients until cervical dilation is performed. The physician needs a means to determine where to place the catheter in order to assure placement of the distal tip of the balloon far enough past the inner os to prevent the uneven resistance from pushing the balloon out of the cervix and to prevent extension far enough to injure the uterine wall. Use of a fixed-length balloon without means of distal anchor will not work, since it will not be possible to maintain sufficient forward pressure on a flexible catheter across the vaginal canal to prevent the cervix from ejecting the balloon.

Another obvious solution would be the use of two inelastic balloons adjacent to one another. In this embodiment, a distal anchor balloon would be placed and inflated in the uterus to hold the catheter in place while a proximal balloon is inflated to dilate the cervical canal. Because inelastic balloons require a taper in order to be folded and a space to be glued to the catheter body, there will be a gap between the two balloons, potentially causing the distal portion of the cervical canal to not be adequately dilated. If the catheter is alternately pulled so an ellipsoidal balloon is located at the point of the inner os, the ellipsoidal balloon may over dilate a portion of the cervix, causing damage such as tearing or cervical incompetence, with resultant fetal loss.

Another obvious solution would be to create a single inelastic balloon that has two sections, the proximal section cylindrical and the distal balloon ellipsoidal in shape. In the single balloon embodiment, the ellipsoidal portion of the balloon is assumed to inflate before the cylindrical section based on the lack of external pressure in the uterus. The feature assumes the catheter is placed correctly and that the uterus is flexible and uniform. The inelastic ellipsoidal balloon is intended to act as an anchor and to dilate the internal os. This embodiment poses a potential problem in that the wider portion of the ellipsoidal section of the balloon could be positioned within the cervix causing over dilation and resultant damage.

A continuing need exists for a cervical canal dilator including a dilating balloon that dilates the entire length of the cervical canal to a predetermined diameter and which has a means for positioning the dilating balloon in patients with differing internal geometries so that it reliably dilates the full length of the cervical canal without being displaced.

### SUMMARY OF THE INVENTION

A cervical canal dilator is described which includes an elongate tubular shaft having an outer surface, a distal end portion, and a proximal end portion. The distal end portion and the proximal end portion define a first longitudinal axis. The shaft defines at least two internal lumens including a first internal lumen and a second internal lumen aligned with the longitudinal axis. The distal end portion defines a tapered tip.

A first inflatable member is positioned on the outer surface of the distal end of the shaft. The first member is in fluid communication with the first lumen and is configured to be positioned between a deflated position and an inflated position. The first member is fabricated of either an elastic material to minimize the profile when in a deflated position or an inelastic material to maximize strength when in an inflated position.

A second inflatable member, which is longer than the first member, is positioned on the outer surface of the distal end portion of the shaft so that it completely covers the first inflatable member. The second member is in fluid communication with the second lumen and is configured for being positioned between a deflated position and a predetermined maximum diameter of inflation. The second member is fabricated of a non-elastic material configured to limit the inflation of the second member to the predetermined diameter of maximum inflation.

A control system is connected with the at least two lumens and includes potential means for monitoring the pressure.

The solution envisioned herein is the placement of one balloon contained completely within a longer second balloon. Since the first balloon can not expand wider than the intended dilation, there is no potential for excessive dilation of any portion of the cervical canal.

In this embodiment, the catheter is inserted through the cervix far enough that the user considers the internal balloon, which is within the distal portion of the external balloon, to be situated within the uterus. This distance is estimated based on the expected length of the cervix and can be aided by the inclusion of markings on the balloon or shaft indicating the distance from the tip. If the catheter is not positioned far enough through the cervix, the balloon will not inflate at low pressure, such as available by manual compression of a standard syringe. If the catheter is positioned so that a portion of the internal balloon is located within the uterus, inflation of the balloon will cause the catheter to slide the desired distance into the uterus positioning the distal end of the outer balloon a set distance past the inner os and temporarily anchoring the catheter for further inflation. If the catheter is positioned so that the internal balloon is located entirely within the uterus, the balloon will inflate and the catheter can be pulled backward to position the outer balloon a set distance past the inner os. When the external balloon is inflated with a high pressure syringe, such as those used to inflate angioplasty balloons, using a compressible gas or an incompressible fluid, the balloon will initially expand in the uterus and outer os, where resistance is lowest, then incrementally inflate within the internal os where resistance is highest. This incremental inflation will hold the balloon in place while the entire cervical canal is dilated.

A third balloon can be located within the proximal end of the second balloon, with a distance between the first and third balloons sufficient to accommodate the length of the cervix. In this design, the inflated internal balloons could hold the respective ends of the external balloon in the uterus and vagina at the start of dilation.

In one embodiment, the internal balloon(s) are comprised of an elastic or compliant material, such as silicone. In another embodiment, the internal balloon(s) are comprised of an inelastic or noncompliant material. This later embodiment provides increased strength and dependability of inflation diameter with a set volume of gas or liquid and allows the balloon to be inflated with higher pressures to aid in securing its position within the uterus.

Because non-elastic balloons must be folded to minimize their profile upon deflation, a new manufacturing process is required. This process allows the internal balloon or balloons to be folded tightly and to maintain the tight fold while the outer balloon is placed and bonded over the internal balloon while making the catheter as small a diameter and as smooth as possible for insertion into the body.

One embodiment envisioned herein is to place a thin elastic tube over the folded internal balloon to hold it tight so the outer balloon can be applied and folded over the internal balloon. This thin elastic tube would be manufactured to break under pressures lower than those used to inflate the balloon. A second method is to place or tie bands around the internal balloon which will stretch or break under a set pressure, thereby releasing the balloon from the folded state upon inflation.

In one embodiment of the envisioned invention, incremental bands of differing strengths can be used to cause the internal balloon to inflate in sections, such as from the os toward the uterus to anchor the balloon within the os, or from the distal end first to cause the balloon to be squeezed into the uterus, thus assuring that the distal end of the dilating balloon is past the inner os. Still another embodiment would use thin elastic tubes or bands which would stretch to the full internal diameter under pressures lower than those to be used to inflate the balloon.

The external balloon would need to be placed over the internal balloon after the internal balloon is attached to the catheter body. In previous use, a non-compliant balloon is molded to have edges which are tapered to have internal diameters that match the external diameter of the catheter shaft to which they are to be attached. In order to be placed over another balloon, this design would require that one edge of the external balloon be tapered to a diameter wide enough to be passed over the affixed internal balloon. In order to effect a seal at the balloon edge with the wider taper, the surface of the catheter will need to be modified to create a wider edge to which to bond the outer balloon. This could be accomplished by affixing a wider tube which has an inner diameter equal to the outer diameter of the catheter shaft and an outer diameter equal to the inner diameter of the balloon taper; this tube would preferable be made of inelastic material to provide the bond strength required to withstand inflation pressures of 150 psi or higher.

If the inner balloon is made of inelastic material, elastic bands or breakaway ties could be used to hold the deflated balloon in a folded manner while the outer balloon is placed and folded over it. This will minimize the diameter of the catheter for insertion.

### BRIEF DESCRIPTION OF THE DRAWINGS

The following description of a preferred embodiment of the present invention will be better understood when read in conjunction with the appended drawings. It should be understood, however, that the invention is not limited to the precise arrangements shown.

FIG. 1A is a side view of a distal end portion of one preferred embodiment of a cervical canal dilator in a first position constructed in accordance with the present disclosure.

FIG. 1B is a side view of the distal end portion of the cervical canal dilator of FIG. 1A with a sheath in place over the inflatable members.

FIG. 2 is a cross-sectional view along lines 2-2 of the cervical canal dilator of FIG. 1A.

FIG. 3A is a side view of the distal end portion of the cervical canal dilator of FIG. 1A with a first inflatable member in the second position constructed in accordance with the present disclosure.

FIG. 3B is a side view of the distal end portion of the cervical canal dilator of FIG. 1A with the first inflatable member and a second inflatable member in the second position constructed in accordance with the present disclosure.

FIG. 4A is a cross-sectional view along lines 4A-4A of the distal end portion of a second embodiment of the cervical canal dilator of FIG. 1 constructed in accordance with the present disclosure.

FIG. 4B is a cross-sectional view along lines 4B-4B of the distal end portion of the second embodiment of the cervical canal dilator of FIG. 4A constructed in accordance with the present disclosure.

FIG. 4C is a cross-sectional view along lines 4C-4C of the distal end portion of the second embodiment of the cervical canal dilator of FIG. 4A constructed in accordance with the present disclosure.

FIG. 5 is a side view of a cervical canal of a patient and the cervical canal dilator of FIG. 1A in the first position.

FIG. 6 is a side view of the cervical canal and the cervical canal dilator of FIG. 1A with a first inflatable member in the second position.

FIG. 7 is a side view of the cervical canal and the cervical canal dilator of FIG. 1A with the first inflatable member and a second inflatable member in the second position.

FIG. 8 is a side view of a distal end portion of a third embodiment of the cervical canal dilator of FIG. 1A, without the balloons attached, constructed in accordance with the present disclosure.

FIG. 9 is a side view of a distal end portion of one preferred embodiment of a cervical canal dilator of FIG. 1A constructed in accordance with the present disclosure.

### PREFERRED EMBODIMENT OF THE INVENTION

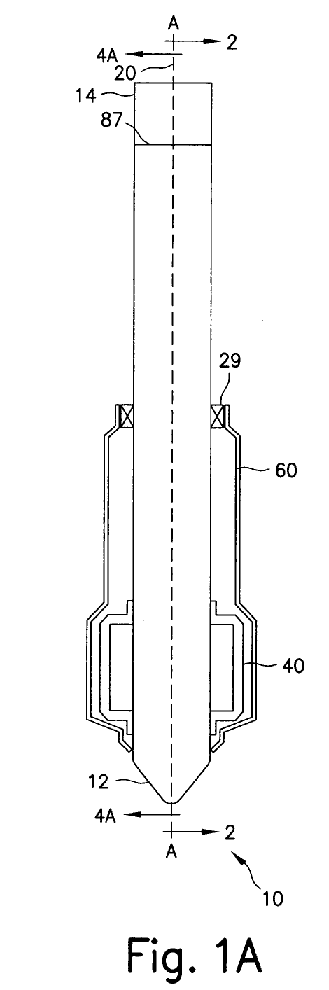

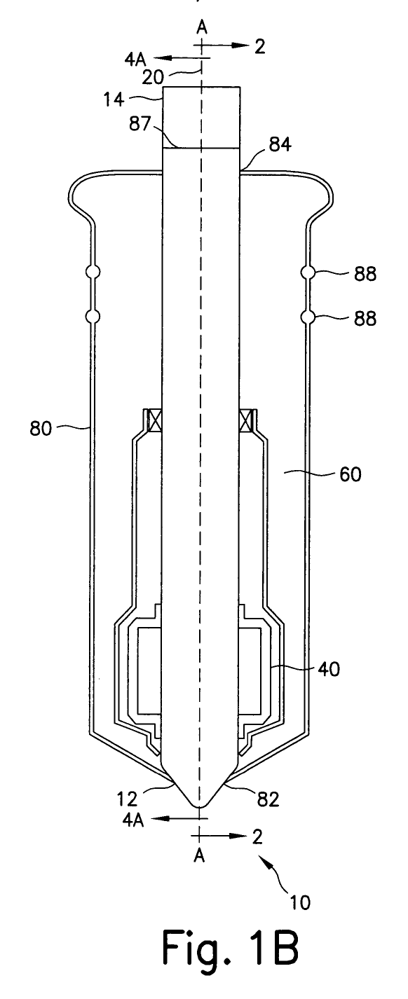

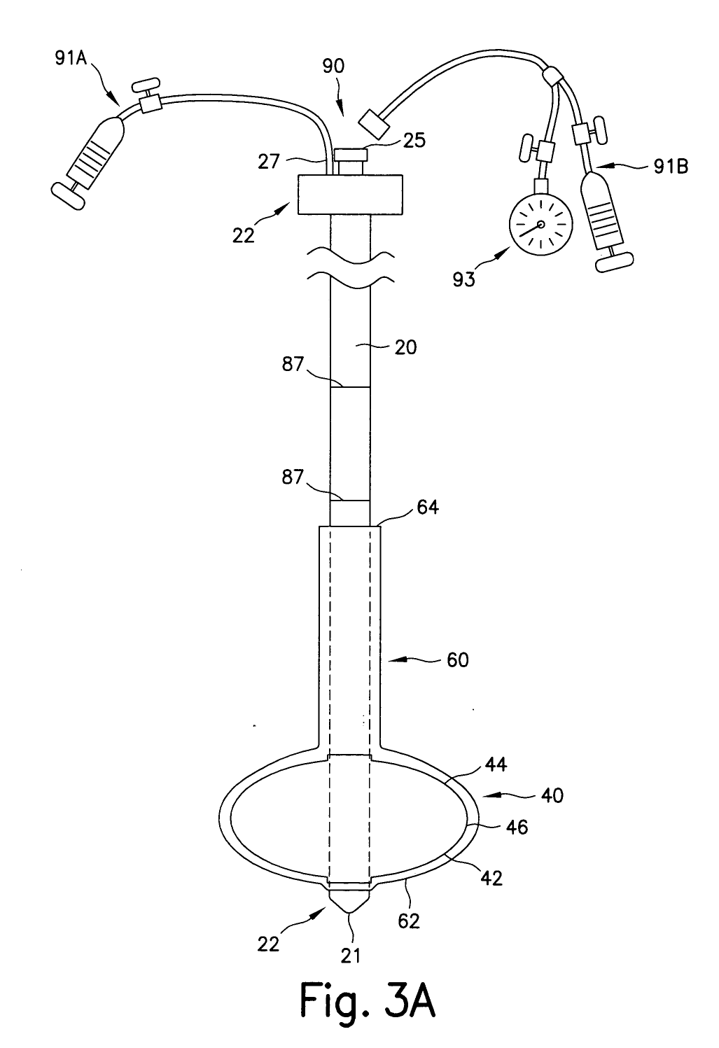

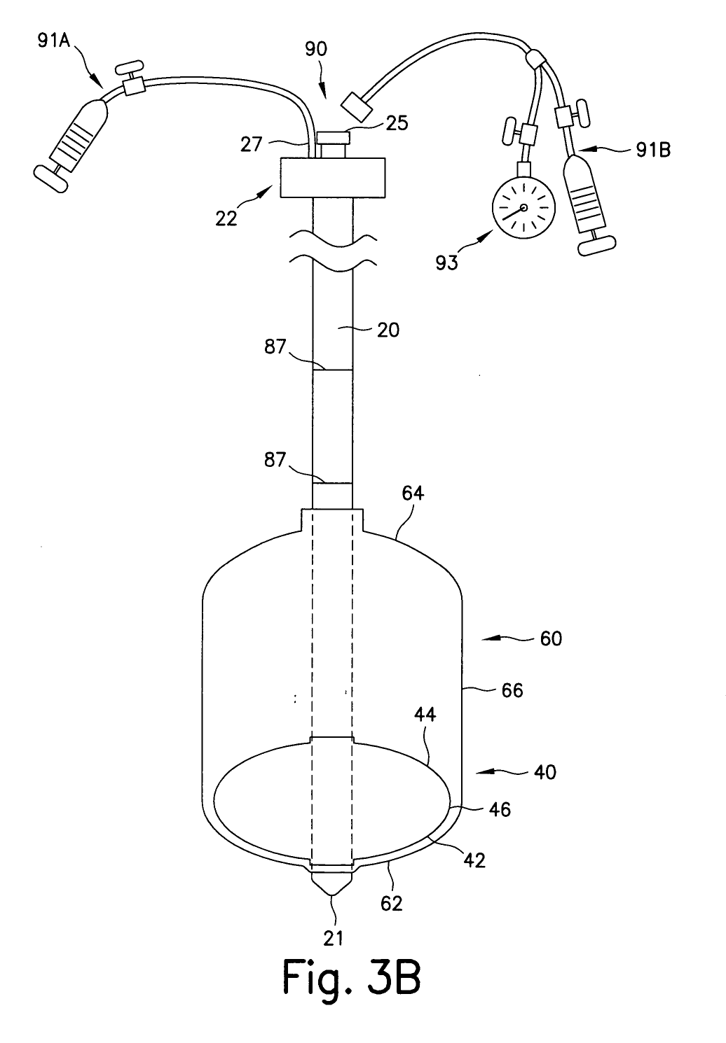

Referring now in specific detail to the drawings in which like referenced numerals identify similar or identical elements throughout the several views, and initially to FIG. 1A, a novel cervical canal dilator assembly 10 is shown having a shaft 20, a first inflatable member 40, a second inflatable member 60 and a control system 90 (see FIG. 3A). Cervical canal dilator assembly 10, hereinafter referred to as “dilator 10” has a distal end 12 and a proximal end 14 defining a longitudinal axis-A.

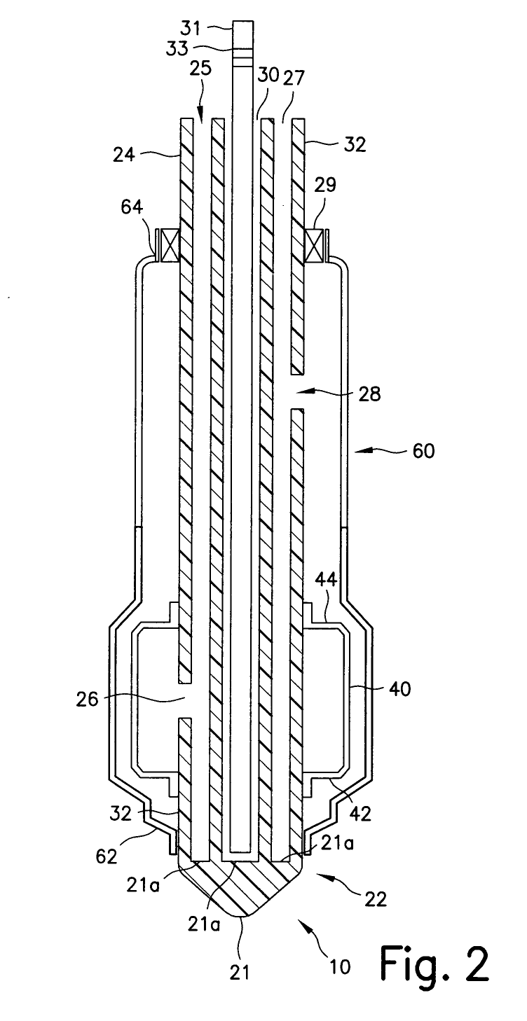

Referring now to FIGS. 1A, 1B, 2, 3A, 3B, 3C, 4A and 4B, shaft 20 has a distal end portion 22 and a proximal end portion 24 aligned with first longitudinal axis-A. Distal end portion 22 includes a tip 21 having a solid circular base 21a and a tapered or conical outer shape. Tip 21 is advantageously shaped for ease of insertion into the cervical canal of the patient. Shaft 20 has a cylindrical outer surface 32. Proximal end portion 24 is connected with control system 90.

Shaft 20 is fabricated of a medical grade plastic or composite material. Shaft 20 can have a flexible, semi-rigid, or rigid configuration. Flexible shaft 20, in one preferred embodiment, is highly flexible to the point of becoming at least partially floppy and having only a moderate degree of stiffness along longitudinal axis-A. The rigid construction can be straight or include an arcuate portion encompassing at least part of distal end portion 22. The semi-rigid configuration is flexible and/or bendable such that semi-rigid shaft 20 can retain a specifically defined shape. The term flexible shaft 20, as noted herein, refers to shafts 20 having flexible configurations selectively augmented by a wire for shaping and/or stiffening.

Shaft 20 is a solid shaft 20 defining inner walls for a first lumen 25, a second lumen 27, and a third lumen 30. First lumen 25 is connected with a port 26 positioned through outer surface 32 for communication with first inflatable member 40. Similarly, second lumen 27 is connected with a port 28 positioned through outer surface 32 for communication with second inflatable member 60. Lumens 25, 27, and 30 are terminated and sealed on their distal ends by base 21a of tip 21 and connected with control system 90 on their opposing proximal ends.

Third lumen 30 is preferably positioned between lumens 25 and 27 and axially aligned with the longitudinal axis-A. Third lumen 30 can be configured to only define a proximal end port connected with control system 90 or to define a side port 70 in outer surface 32 (see FIG. 10) distal to first member 40 and proximal to tip 21. Port 70 allows communication with the inside of the uterus in applications, such as for example, prior to completion of cervical dilation and removal of the dilator. Uses of this channel can also include diagnostic readings or infusion of therapeutic agents to treat pain and bleeding within the uterus.

Flexible shaft 20 includes a wire 31, an elongate element, suitably sized for positioning in one of the lumens of shaft 20. When wire 31 is positioned in one of the lumens of shaft 20, a distal end of wire 31 abuts base 21a and a proximal end extends from shaft 20. Wire 31 is configured for ease of removal and replacement in one of the lumens. Wire 31 provides an improved degree of stiffness along longitudinal axis-A of flexible shaft 20. In addition, wire 31 assists in the shaping of the highly flexible configuration of shaft 20.

In one preferred embodiment, wire 31 is positioned in lumen 30. In another preferred embodiment, wire 31 is positioned in the first lumen 25 or second lumen 27. When wire 31 is positioned in lumen 25 or 27, wire 31 is preferably removed prior to the application of fluid or gas to the lumens, but wire 31 can be selectively retracted at any time prior to the application of the fluid or gas to lumen 25, 27, or 30.

Wire 31 is made of a bendable material with a capable of holding an altered shape such that shaft 20 can be shaped for insertion in a cervix oriented at an angle to the patient's vagina. Shaping wire 31 and/or shaft 20 includes bending at least the distal end portion 22 of shaft 20 to replicate the approximate angle between the cervical opening and the vaginal canal such that the distal end portion is generally perpendicular to the cervical opening. Wire 31 could also be preformed to have an arcuate shape or arcuate bend based on a shapeable material. Arcuate bent wire 31 is retractable from shaft 20 so that the flexibility of shaft 20 can be selectively controlled when shaft 20 is a flexible member.

The stiffness of flexible shaft 20 and in particular, distal end portion 22, can be controlled by partially withdrawing or retracting wire 31 from contact with base 21a and/or distal end portion 22. In this manner, when distal end portion 22 is positioned inside the cervical canal, for example, distal end portion 22 can be made less stiff than the portion of shaft 20 inside the vagina of the patient. Wire 31 can be selectively retracted so the portion of shaft 20 distal to member 40 or distal to member 60, for example, is more flexible than the remaining proximal portions of shaft 20. Wire 31 can be adjustably bent along its full length to bend shaft 20 in an at least partially arcuate shape that includes, for example, a purely arcuate shape or a combination of angled and arcuate shapes prior to or after positioning wire 31 in shaft 20. Wire 31 is retracted to predetermined positions within shaft 20 using markings 33 positioned on the proximal end of wire 31. Wire 31 is preferably made of a medical grade metal and retains its axial stiffness while bent.

First inflatable member or member 40 is positioned proximal to and in juxtaposition with tip 21 and has a compressed or folded annular shape in a first position. Member 40 has a proximal end 44 and a distal end 42 and is fabricated of a stretchable or non-stretchable medical grade rubber, plastic, or composite material suitable for uterine applications. In one preferred embodiment, when a gas or fluid is supplied by control system 90 through lumen 25 to member 40, member 40 expands to a cylindrical outer diameter 46.

The length of member 40 will be in the range of 1 to 4 cm to minimize the portion of the dilator assembly positioned within the uterus. This will prevent the device from damaging the edges of the uterus when the uterus is oriented at an angle to the cervix.

First inflatable member 40 can be inflated to a desired diameter by means for a system 91A supplying a controlled amount or pressure of fluid or gas, the use of means for measuring pressure, or combinations thereof.

Second inflatable member or member 60 is positioned over member 40 so that it completely covers member 40 and has a compressed or folded annular shape in the first position. The first position for members 40 and 60 is a compact position intended to minimize the dimension in the radial direction from the longitudinal axis-A. Member 60 has a distal end 62 and a proximal end 64. Member 60 is fabricated of a non-stretchable or non-elastic type medical grade plastic or composite material suitable for internal applications. Member 60 can have a membrane or a woven configuration. When a gas or fluid is supplied by control system 90 through lumen 27 and the port to member 60, member 60 inflates both radially and axially into an annular shape having a cylindrical outer surface 66 generally parallel to the longitudinal axis-A. Distal end portion 62 and proximal end portion 64 have generally tapered spheroid shapes.

Member 60 comes in a range of predetermined maximum diameters of inflation such as, but not limited to 4 mm to 20 mm. The length of member 60 is suitable for extending at least the length of a cervical canal of the patient. Member 60 is configured for uniformly inflating along its length such that the cervix is uniformly dilated as member 60 is inflated to its predetermined maximum diameter of inflation. The predetermined maximum diameter of inflation or maximum inflatable diameter is defined herein as the diameter defined by the configuration of non-elastic second member when fully inflated.

Second member 60 is fabricated and/or constructed of non-elastic material having sufficient strength such that upon reaching its predetermined maximum diameter at full inflation, additional pressure communicated to member 60 will increase the pressure within member 60, but the diameter of member 60 remains fixed. The fixed maximum diameter along the axis perpendicular to longitudinal axis-A of member 60 also functions to reduce the risk of over expanding the cervical canal due to over inflation of the dilating member. Member 60 has a suitable length to ensure it will encompass the full length of a patient's cervical canal.

Because member 60 will not expand beyond the predetermined diameter, cylindrical outer surface 46 of member 40 will not be wider than cylindrical outer surface 66 of member 60.

The length of member 60 will preferably be in the range of approximately 4 cm to 8 cm to dilate the full length in the majority of female cervixes while minimizing the portion of member 60 inflated within the vagina where it could interfere with visualization of the outer edge of the cervix and to monitor the progress of dilation.

Sheath 80 is a thin layer of medical grade low outer surface friction plastic material having a first position at least partially covering dilator assembly 10 including member 60, and at least the distal end portion 22 of shaft 20. Sheath 80 can be a shrink wrapped layer or a loosely conforming layer, for example. Sheath 80 has a distal end 82 and a proximal end 84. Distal end 82 is positioned over tip 21 and can include perforations, serrations, or indentations to facilitate sheath 80 in stretching or splitting so that it can be removed from shaft 20 and member 60.

Distal end 82 is configured to stretch or at least partially separate into segments upon the retraction of sheath 80 proximally such that tip 21 extends through distal end 82 and second member 60 is selectively retracted or uncovered from sheath 80. Sheath 80 is adapted to be retracted proximally along longitudinal axis-A. Sheath 80 can be coated with a lubricating material suitable for uterine applications such as a hydrophilic material to allow for an easy and rapid insertion into the cervical opening.

Sheath 80 has markings 88 positioned at intervals along proximal end 84 to indicate the depth of penetration of tip 21 and member 60 into and/or through the cervical canal. Additional markings 87 on shaft 20, for example, indicate how far sheath 80 has been retracted and to thereby ensure that sheath 80 is clear of member 60, for example, prior to inflation.

Control system 90 includes means for a system 91B to deliver fluid or gas. Means for a system 91B can include items typically found in pressurized fluid systems such as, but not limited to a closed circuit of lines, connectors, valves, supply and exhaust reservoirs, pumps, pressure gauges, and safety devices such as pressure release valves. Means for a pressure system 91B includes separate pressure systems for first member 40 and second member 60. Items such as the reservoir and pump, for example can be a single item such as a syringe having suitable fluid capacity or separate items.

Means for a system 91B includes means for measuring pressure 93, such as a pressure gauge, in communication with second inflatable member 60. Means for measuring pressure 93 is configured to measure the pressure within second member 60, and by relation, pressure within the cervical canal. The ability to measure the pressure within member 60 allows the physician to have an improved method of control over the dilation process and determining when the maximum dilation of second member 60 is achieved. Means for measuring pressure 93 and means for a system 91B are configured for precisely controlling the amount of pressure applied to member 60 and thereby to the patient during the dilation process.

Means for measuring pressure 93 can include an adjustable tolerance or range setting such that if the pressure drops below or rises above a particular range then an alarm or warning is provided. It is further envisioned that the pressure in member 60 can be made to increase or decrease at a predetermined rate by increments over time.

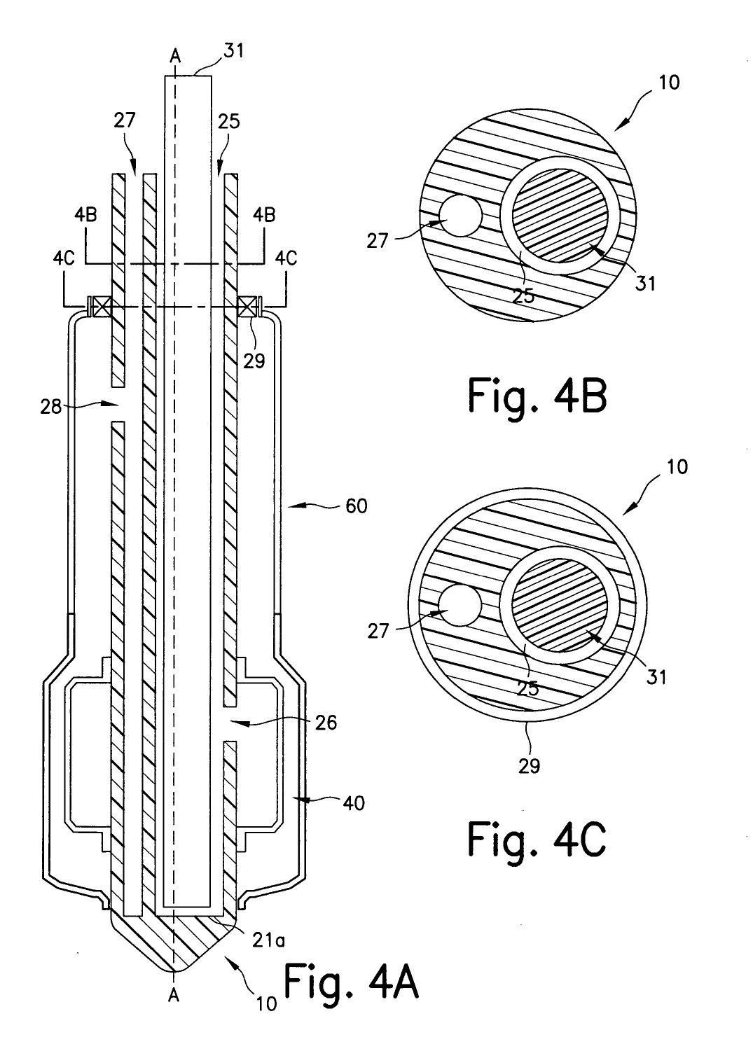

Referring now to FIGS. 3A, 3B, 4A, 4B and 4C, in another preferred embodiment, cervical canal dilator 10 has a shaft 20 including only first internal lumen 25 and second internal lumen 27. First lumen 25 has a diameter suitable for being in communication with first member 40 through port 26 and for the positioning of wire 31 when shaft 20 is in the flexible configuration. Second lumen 27 is in communication with the second member 60 through port 28. In this embodiment, the distal end of wire 31 is initially positioned abutting base 21a and the proximal end extends from shaft 20. Wire 31 functions to provide an improved element of stiffness axially, along the longitudinal axis-A. Wire 31 is readily positioned and removed from lumen 30, but is removed prior to the connecting first lumen 25 with control system 90 for communication. In the rigid or semi-rigid configurations not requiring wire 31 to augment stiffening of shaft 20, lumens 25 and 27 are not necessarily sized for the positioning of wire 31. Further, if it is desired that member 40 is expanded before retraction of wire 31, lumen 27 can be designed to accommodate wire 31.

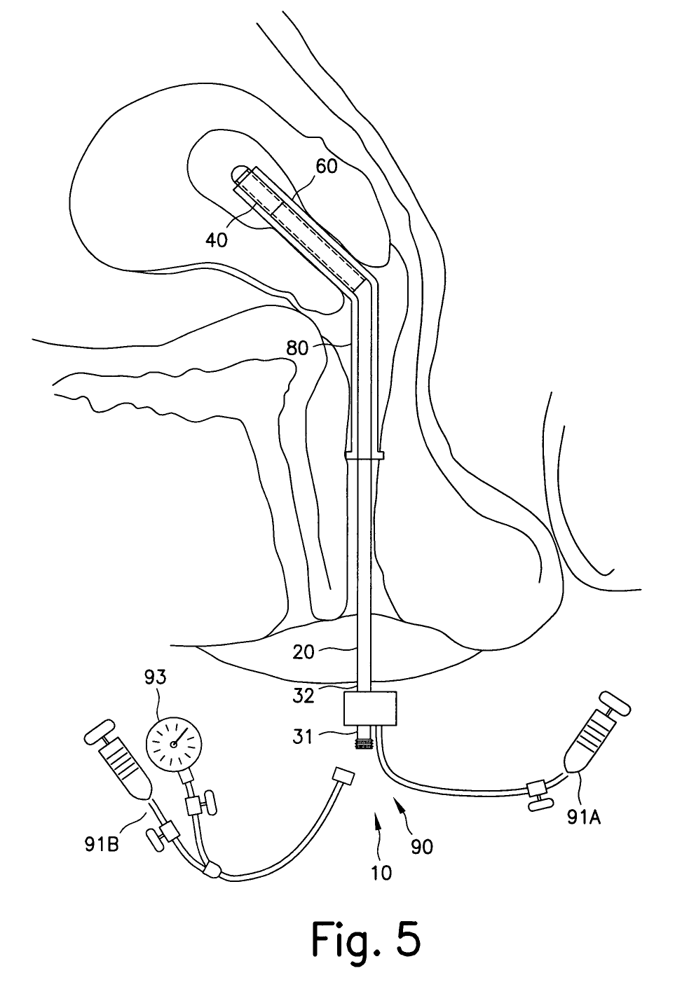

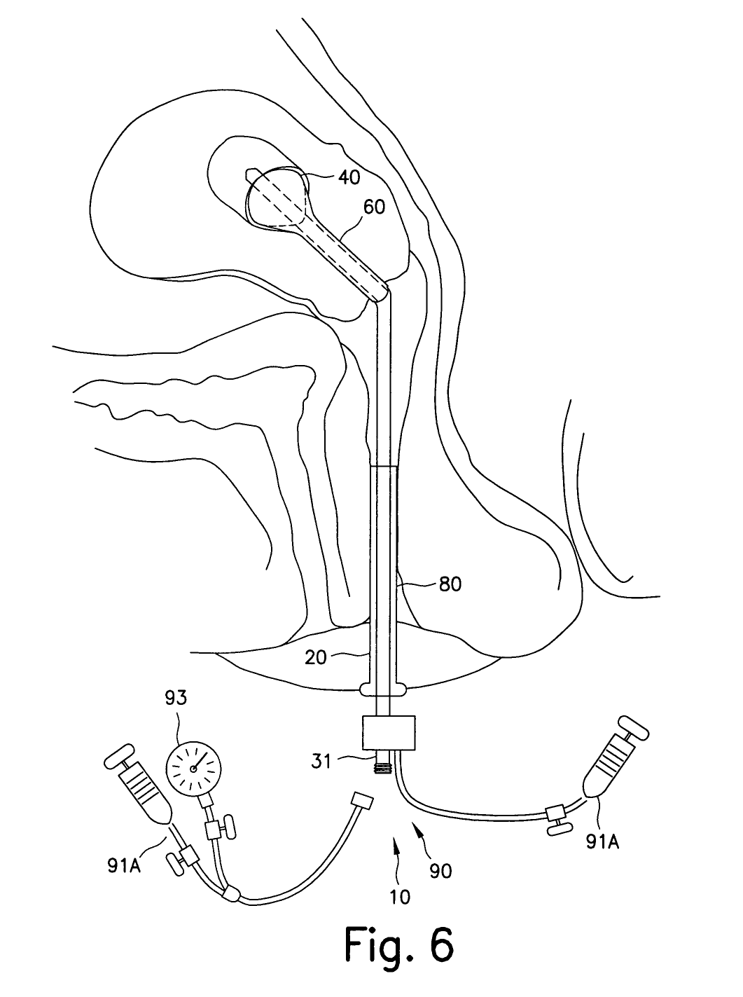

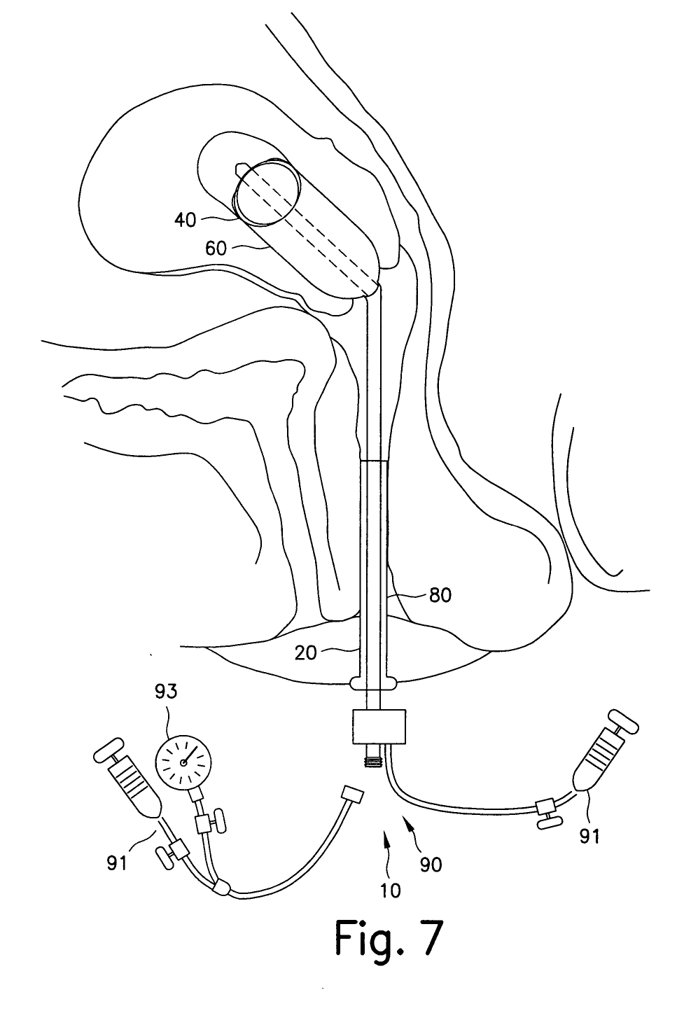

As shown in FIGS. 1A, 1B, 2, 3A, 3B, 4A, and 5-7, in operation, the physician selects a desired configuration of cervical canal dilator 10 for application with the patient for the dilation of the patient's cervical canal to the predetermined maximum diameter. This process can include evaluating the patient internal geometries, such as the angle between the vagina and the cervical opening, to determine whether the rigid, semi-rigid, or flexible shaft 20 configuration will be utilized. Cervical canal dilator assembly 10 is initially in the first position with first member 40 and second member 60 compactly positioned against outer surface 32. Depending upon the configuration, cervical canal dilator 10 is covered by sheath 80 compactly positioned against outer surface 32 and second member 60.

When utilized, wire 31 positioned in one of the lumens such that the distal end of the wire abuts base 21a and the proximal end of the wire extends from shaft 20 to provide accessibility to the physician. When wire 31 is made of bendable material, it can be shaped to the desired angle or arcuate orientation before or after positioning in flexible shaft 20. The bending of wire 31 is preferably performed when dilator 10 is in the first position with wire 31 positioned fully in shaft 20 such that the distal end of wire 31 is abutting base 21a. Wire 31, for example, can be shaped for insertion in a cervix that is oriented at an angle to the patient's vagina.

Dilator 10 in this position has a diameter less than 4 mm and is considered suitable for application in all cervixes. Dilator 10 is positioned at least partially into the cervical opening. Once distal end 12 has been inserted a predetermined distance, such as approximately 4 mm, into the cervix, wire 31 when present can be selectively retracted from shaft 20 such that when tip 21 is positioned inside the cervical canal, distal end 22 can be made advantageously less stiff than the portion of shaft 20 inside the vagina. The reduction in stiffness can reduce the risk of accidental damage to the cervix.

Distal end 12 is then inserted further into the cervix a second predetermined distance, such as for example three centimeters, to position first inflatable member 40 within the uterus. With wire 31 removed, shaft 20 retains sufficient axial rigidity for forwarding through the cervical canal and yet is suitably flexible or floppy to drastically reduce the likelihood of inadvertently perforating the uterine wall. The penetration through the cervical canal can be aided by a hydrophilic material, positioned on tip 21 or the surface of sheath 80. When configured with sheath 80, markings 88 positioned at intervals along proximal end 84 indicate the depth of penetration of tip 21, member 40, and member 60 into and/or through the cervical canal.

When present, sheath 80 is then retracted proximally along longitudinal axis-A to uncover the inflatable members. Using control system 90, means for a system 91A sends a predetermined volume of fluid or gas, such as, but not limited to a saline solution or carbon dioxide, to inflate member 40 and initiate placing dilator 10 from the first position to the second position. A syringe or another pressurizing and reservoir system can be used to inflate member 40. Dilator 10 is then moved proximally until member 40 engages the internal edge of cervix.

Control system 90, including means for a system 91B and means for measuring pressure 93, is used to inflate second member 60 from the first position to the predetermined maximum diameter of inflation or second position. Member 60 expands both axially and radially initially in a uniform manner-into an elongate cylindrical shape having spheroid distal and proximal ends.

The inflation of member 60 continues after the axial limit is reached in a uniform radial inflation until the predetermined maximum diameter of inflation is achieved. This advantageously uniformly inflates the diameter such that a uniform pressure is placed along the cervix and limits the dilation of the cervix to the desired diameter. The inflation of member 60 is typically done in a series of graduated steps and is completed by the positioning of dilator 10 in the second position. A syringe or another pressurizing and reservoir system can be used to inflate member 60. Means for measuring pressure such as a pressure gauge 93 is preferably used to monitor the pressure applied to the second member and cervix during the dilation process and determines when the cervix has expanded in response to the pressure of the second member by a reduction in pressure, for example, as well as determining when second inflatable member has reached its maximum diameter of inflation or second position.

Means for measuring pressure 93 can also be advantageously used to measure the dilation or relaxation of the cervix after an incremental increase of the pressure to second member 60 for dilation. Alternatively, or in combination, member 60 could be inflated to its maximum diameter using a predetermined amount of fluid or gas.

When second member 60 is inflated to its maximum predetermined diameter of inflation and the desired cervical canal dilation is achieved, first member 40 and second member 60 are deflated returning dilator 10 to the approximate diameter of the first position. Dilator 10 is then withdrawn from the patient. In an alternate use, the catheter is withdrawn without deflation of the balloons, demonstrating desired dilation of the full length of the cervix.

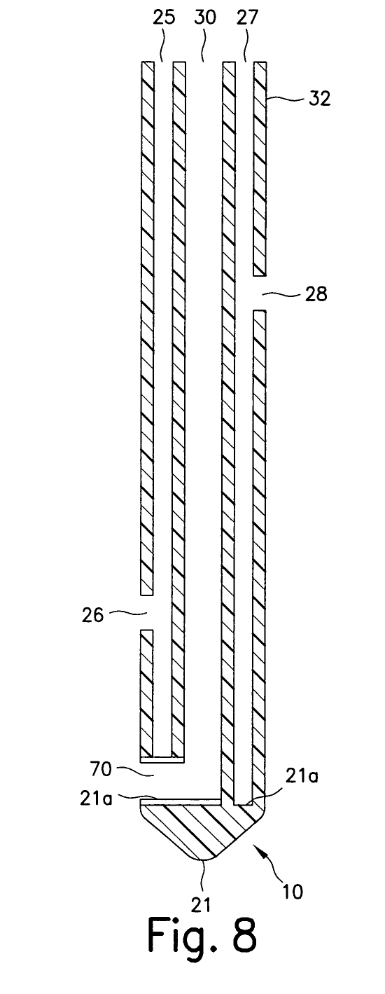

In another preferred embodiment, as shown in FIG. 8, cervical canal dilator 10 includes a side port 70 defined in outer wall 32 of third lumen 30. Lumen 30 is in communication with control system 90. Port 70 is preferably distal to the inflatable member 60, allowing communication with the inside of the uterus for applications such as but not limited to providing treatment inside the uterus prior to completion of cervical dilation and removal of the catheter. Port 70 also accommodates, for example, the making of diagnostic readings from the inside of the uterus that can be recorded using control system 90. Alternatively, therapeutic agents can be injected through port 70 into the uterus to treat conditions such as cramps or bleeding. Port 70 is in outer surface 32 in order to not interfere with the streamlined low friction shape of tip 21.

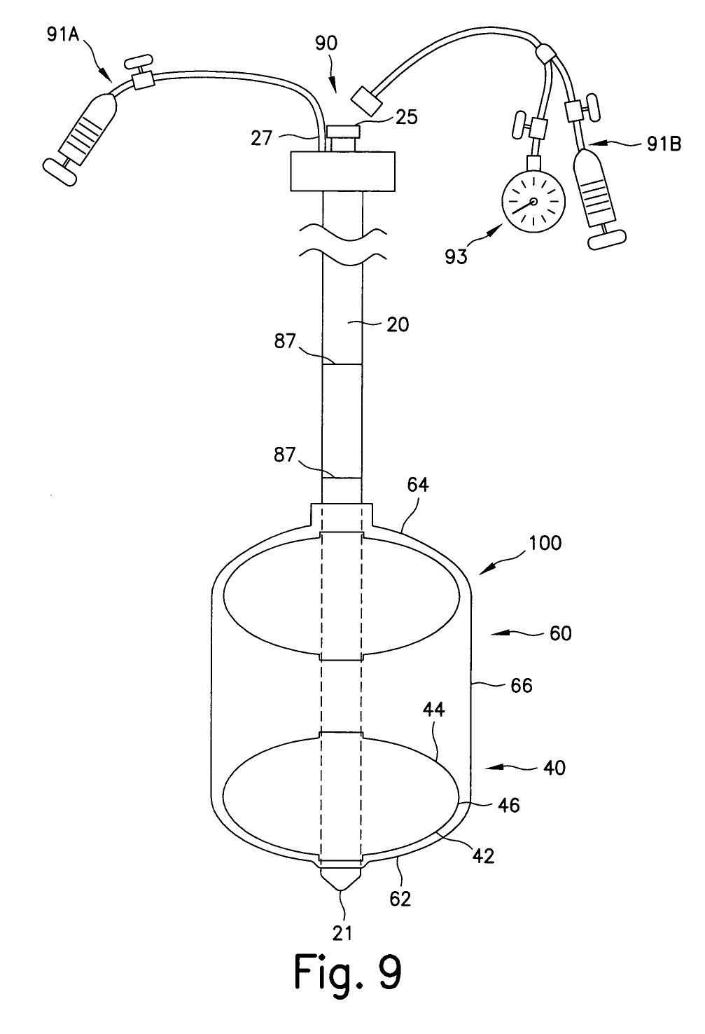

FIG. 9 shows a further embodiment of cervical canal dilator 10 including a third expandable member 100 enclosed within member 60, proximal to the center point of member 60. In this embodiment, expandable members 40 and 100 are expanded to anchor member 60 to both edges of the cervical canal prior to inflation of member 60.

Although the illustrative embodiments of the present disclosure have been described herein with reference to the accompanying drawings, it is to be understood that the disclosure is not limited to those precise embodiments, and that various other changes and modifications may be affected therein by one skilled in the art without departing from the scope or spirit of the disclosure. All such changes and modifications are intended to be included within the scope of the disclosure.

What is claimed:

1. A cervical canal dilator comprising:
an elongate tubular shaft having an outer surface, a distal end portion, and a proximal end portion, the distal end portion and the proximal end portion defining a first longitudinal axis, the shaft defining at least two internal lumens including a first internal lumen and a second internal lumen aligned with the longitudinal axis, the distal end having a tapered tip;
a first inflatable member positioned on the outer surface of the distal end portion of the shaft, the first member being in communication with the first lumen, the first member being positionable between a deflated position and an inflated position;
a second inflatable member positioned on the outer surface of the distal end portion of the shaft, said second inflatable member being longer than said first inflatable member and completely covering said first inflatable member, said second member being in communication with said second lumen, said second inflatable member being positionable between a deflated position and a predetermined maximum diameter of inflation, said second member being fabricated of a non-elastic material configured to limit the inflation of the second member to the predetermined maximum diameter of inflation; and
a control system connected with the at least two lumens and including inserting means for inserting a fluid or gas into said at least two lumens, the inserting means for the fluid or gas system being in communication with the at least two lumens.

2. The cervical canal dilator of claim 1, wherein the first inflatable member is fabricated of a non-elastic material configured to hold a predetermined shape when in an inflated position.

3. The cervical canal dilator in claim 2, wherein the first inflatable member is affixed with elastic or breakable bands to minimize its profile while the second inflatable member is placed over the first inflatable member.

4. The cervical canal dilator of claim 1, wherein the first inflatable member is fabricated of an elastic material configured to present a minimal profile when in a deflated position.

5. The cervical canal dilator of claim 1, wherein there are at least two first inflatable members.

6. The cervical canal dilator of claim 5, wherein each inflatable member is in communication with a unique lumen.

7. The cervical canal dilator of claim 5, wherein the first inflatable members are in communication with a common lumen.

8. The cervical canal dilator of claim 1, wherein the second member is tapered at one edge to a diameter that is at least as wide as the outer diameter of the first member when it is attached to the catheter shaft and the catheter shaft is modified to have a wider diameter at one edge for bonding of the second member

9. The cervical canal dilator of claim 1, wherein the shaft is a flexible shaft and a removable wire is positionable in one of the lumens, the wire providing increased stiffness to aid insertion of the catheter.

10. The cervical canal dilator of claim 9, wherein the shaft includes two lumens and at least one of the lumens defines a passageway suitable for the positioning of the wire.

11. The cervical canal dilator of claim 9, wherein the shaft has at least three lumens, a third lumen defining a passageway parallel to the longitudinal axis and suitable for the positioning of the wire.

12. The cervical canal dilator of claim 1, wherein the control system includes means for measuring pressure within the second member.

13. The cervical canal dilator of claim 1, wherein the surface of the second member is covered with an agent to prevent the second member from moving laterally during inflation or the surface of the second member is textured or has an elastic textured material stretched over it, to grip the walls of the cervical canal to prevent the second member from moving laterally during inflation.

14. The cervical canal dilator of claim 1, wherein the length of the second member is in the range between 4 and 8 cm and the predetermined maximum diameter of inflation of the second member is from 4 mm to 20 mm.

15. The cervical canal dilator of claim 1, wherein the shaft or balloons include markings to determine the distance from the tip of the catheter

16. The cervical canal dilator of claim 1, wherein a sheath is positioned over the second member, the sheath being retractable.

17. The cervical canal dilator of claim 16, wherein the sheath includes a coating of a lubricating material suitable for cervical applications.

18. The cervical canal dilator of claim 9, wherein the flexible shaft is bendable into an at least partially arcuate shape, the wire being bendable such that the shaft can be shaped in an at least partially arcuate shape suitable for insertion into a vagina and a cervix of a patient when the cervix is aligned at an angle to the vagina.

19. The cervical canal dilator of claim 18, wherein the removal of the shaped wire will result in a reduction in the radius of the arcuate shaped curve of the shaft.

20. The cervical canal dilator of claim 9, wherein the wire is configured to be selectively positioned within one of the lumens of the shaft, the portions of the shaft including the wire having an increased axial stiffness.

21. The cervical canal dilator of claim 9, wherein markings are positioned on a proximal end of the wire, the markings indicating the position of the distal end of the wire relative to the tip of the shaft and the first and second inflatable members.

22. The cervical canal dilator of claim 10, wherein the passageway of the third lumen is in communication with a port defined in the side of the shaft at a point distal to the first inflatable member, the port being suitable for collecting diagnostic data from the inside of the uterus or infusing the uterus with therapeutic agents.

23. The cervical canal dilator of claim 22, wherein the data collected from the port is used to confirm positioning within the uterus.

24. The cervical canal dilator of claim 12, wherein the means for measuring pressure is a pressure gauge.

25. The cervical canal dilator of claim 1, wherein the tip includes a base, the base being configured to terminate the distal ends of the lumens.

26. The cervical canal dilator of claim 25, wherein the tip of the catheter is made of rounded metal to seal the distal ends of the lumens

27. The cervical canal dilator of claim 1 wherein the length of the second member (inelastic dilating balloon) is covered with an elastic balloon material to protect it during insertion or otherwise modify the surface characteristics of the second member.

28. A method of dilating a cervical canal of a patient, comprising the steps of:
providing a dilator assembly having a flexible shaft, a first inflatable member, a second inflatable member and a control system, the second inflatable member being fabricated of a non-elastic material and configured to define a uniform maximum diameter for dilating the cervix of a patient, the first inflatable member being internal to the second member and configured to inflate with a predetermined volume of fluid or gas, said control system being in communication with the first inflatable member and the second inflatable member;
positioning the dilator assembly in a first position for penetration into a cervical canal of the patient,
making an initial penetration of the cervical canal;
positioning the first inflatable member through the cervical canal;
placing the dilator assembly in the second position by using the control system for inflating the first member;
positioning the inflated first member against the inner side of the cervical canal;
using the control system to gradually inflate the second member to a maximum diameter of inflation defined by the second member, the second member being configured to dilate the cervical canal of the patient to the predetermined maximum diameter of inflation of the second member;
deflating the first member; deflating the second member; and
withdrawing the dilator assembly from the patient.

29. The method of claim 28, wherein the initial forwarding of the dilator assembly into the cervical canal of the patient is performed using a wire.

30. The method of claim 29, wherein the step of positioning further includes bending any portion of the flexible shaft such that the flexible shaft can be shaped to include an at least partially arcuate portion suitable for insertion into a vagina and the cervix of the patient when the cervix is aligned to the vagina at an angle.

31. The method of claim 29, wherein the step of positioning further includes selectively retracting the wire of the shaft to decrease the stiffness of the distal end portion once the tip has been positioned within the cervix.

32. The method of claim 28, wherein the step of placing includes reading a pressure level on a pressure gauge, the pressure level monitoring the maximum pressure within the second member to determine the cervix has dilated to the predetermined maximum diameter and the dilator assembly can be removed.

33. The method of claim 28, wherein the step of providing includes the shaft, the first inflatable member, and the second inflatable member being at least partially covered by a sheath.

34. The method of claim 33, wherein the step of placing further includes retracting the sheath prior to inflating the first member.

35. The method of claim 28, wherein the step of providing includes a shaft having at least three lumens, one lumen defining a passageway parallel to the longitudinal axis and defining a port on the side of the shaft at a point distal to the inflatable members, the port being suitable for collecting diagnostic data from the inside of the uterus and infusing the uterus with therapeutic agents.

36. The cervical canal dilator of claim 35, wherein the port is used to verify positioning of the catheter tip beyond the cervix prior to inflation of the balloons.

37. The cervical canal dilator of claim 1, wherein the shaft is fabricated of semi-rigid material suitable for bending and retaining a specifically defined bent shape.

38. A female urethral dilator comprising:
an elongate tubular shaft having an outer surface, a distal end portion, and a proximal end portion, the distal end portion and the proximal end portion defining a first longitudinal axis, the shaft defining at least two internal lumens including a first internal lumen and a second internal lumen aligned with the longitudinal axis, the distal end having a tapered tip;
a first inflatable member positioned on the outer surface of the distal end portion of the shaft, the first member being in communication with the first lumen, the first member being positionable between a deflated position and an inflated position;
a second inflatable member positioned on the outer surface of the distal end portion of the shaft, said second inflatable member being longer than said first inflatable member and completely covering said first inflatable member, the second member being in communication with the second lumen, the second member being positionable between a deflated position and a predetermined maximum diameter of inflation, and the second member being fabricated of a non-elastic material configured to limit the inflation of the second member to the predetermined maximum diameter of inflation; and
a control system connected with the at least two lumens and including means for inserting a fluid or gas, the means for the fluid or gas system being in communication with the at least two lumens.
